(19) United States
(12) Patent Application Publication
(10) Pub. No.: US 2002/0191670 A1
(43) Pub. Date: Dec. 19, 2002

(54) Infrared radiation ear thermometer and offset method

(76) Inventors: Yu Chien Huang, Hsinchu City (TW); Simon Taso, Hsinchu City (TW); Vincent Weng, Hsinchu City (TW); Charles Chang, Hsinchu City (TW); Kevin Lin, Hsinchu City (TW); Roger Chen, Hsinchu City (TW); Jason Liao, Hsinchu City (TW)

Correspondence Address: PRO-TECHTOR INTERNATIONAL SERVICES, 20775 Norada Court, Saratoga, CA, 95070-3018, US

(21) Appl. No.: 09/877,354

(22) Filed: Jun. 7, 2001

(51) Int. Cl. G01J 5/06; A61B 5/00; G01J 5/08

(52) U.S. Cl. 374/133; 374/130; 600/549; 600/474; 702/131; 374/131

(57) ABSTRACT

An infrared radiation ear thermometer has an optical system, an infrared detector, an ambient temperature sensor, and display unit, a signal processing section. Wherein, the infrared detector further includes an infrared sensor and a temperature reference sensor; the infrared sensor is deposition on the substrate and the temperature reference sensor is mount near the substrate of the infrared detector to convert the infrared signal into an electrical signal and sense the reference temperature separately. The ambient temperature sensor is set in the space near the optical system to detect the fast change of the ambient temperature. The signal processing section receives the signals from these temperature sensors to produce an offset by a mathematical algorithm. The offset is used to correct the temperature reading and maintain a high precision even though the ear thermometer suffers from an extreme temperature change.

### BACKGROUND OF THE INVENTION

1. Field of the Invention

The present invention relates to an ear infrared radiation ear thermometer for reading the clinical temperature of body by inserting in an external ear canal.

2. Description of the Prior Art

All traditional infrared radiation thermometers suffer some inefficient sensitivity to the ambient temperature due to the temperature dependent detector responsibility and unexpected radiation from the optical system like optical-guide. In most infrared radiation thermometers, there is an electronic means of using a reference temperature sensor to compensate the inefficient sensitivity of temperature change. However, if the ambient temperature changes quickly, the compensation is unlikely to track the change in the detector temperature exactly.

According the disclosed infrared clinical thermometer in U.S. Pat. No. 4,895,164, its combination of a radiation detector should be operated in isothermal condition with an optical-guide. This thermometer especially has a heat conducting block that is constructed and configured so as to remain the thermometer in an isothermal state. However, the isothermal condition is not easy to maintain when the ambient temperature changes rapidly. For example, if the thermometer is taken out from a warm room (say 30° C.) to a cold room (say 15° C.), the isothermal condition is destroyed and the processing of reaching the new isothermal state of the thermometer should take up to half hour or an hour even more to ensure the whole instrument comes to equilibrium.

Another radiation clinical thermometer disclosed in U.S. Pat. 5,024,533 has a probe with an optical guide and an infrared detector, a detection signal processing section, a body temperature operating section, and a display unit. The body temperature operating section receives infrared data from infrared detector, temperature-sensitive data from two reference temperature sensors. These temperature-sensitive data is taken into account the temperature equilibrium between the optical guide and the infrared detector so as to accurately calculate body temperature. These two reference temperature sensors are separately fixed on the infrared detector and the optical guide to detect the temperature difference between the infrared detector and the optical guide. In this case, body temperature comes out basing on the temperature difference from the tow reference temperature sensors under a non-isothermal condition. Unfortunately, it is not simple for this case. First of all, the optical guide is not the only one source to cause the measure error by exchanging radiation with the infrared detector. All the optical system including the inner wall of the infrared detector package, the detector window and even the probe itself may have radiance on the detector element that must be correct in order to achieve the accuracy demand under the non-isothermal state. In addition, the ambient temperature changing may be diversely. The ambient temperature may be unsteady both on the changing directions and timing. If the thermometer is susceptible to rapid temperature changes in random position and timing, the temperature difference between the detector and the guide may be canceled out at a transient moment. But the optical system of the thermometer is actually not stayed on the thermal equivalent condition and the compensation value may not be correct.

### SUMMARY OF THE INVENTION

The primary object of the present invention is to provide an infrared radiation ear thermometer. It is used to read the clinical temperature from the external ear canal with high precision and without waiting for the isothermal condition when the thermometer suffers a rapid ambient temperature change.

The other object of the present invention is to provide a more simple and efficient method to produce an offset. The offset is used to eliminate the temperature reading error arose from the difference among the ambient temperature, the infrared detector, and the optical system; therefore, the accuracy of the infrared radiation ear thermometer is maintained.

### BRIEF DESCRIPTION OF THE DRAWINGS

FIG. 1 is a cross-sectional view of the preferred embodiment showing an internal structure of the present invention.

FIG. 2 is a cutaway view of the infrared detector in FIG. 1.

FIG. 3 is block diagram of the present invention showing signals processing.

FIG. 4 is a cross-sectional view of the test and experiment embodiment that has three temperature reference sensors to develop the algorithm for correcting the measure error caused by instantly ambient change.

FIG. 5 is a graph showing the rapid ambient temperature change which the test and experiment embodiment of FIG. 4 suffers.

FIG. 6 is a graph showing the different temperature change curves from the three sensors in the test and experiment embodiment.

FIG. 7 is a graph showing the slope change at different time after a rapid temperature change.

FIG. 8 is a graph showing the test and experiment embodiment suffers two different rapid ambient temperature increasing sections.

FIG. 9 is a graph showing the test and experiment embodiment suffers a rapid ambient temperature increasing section and decreasing section.

FIG. 10 is graph showing the infrared radiation path for the calculation of the offset when the temperature of the detector element irradiation different from that of the optical-guide.

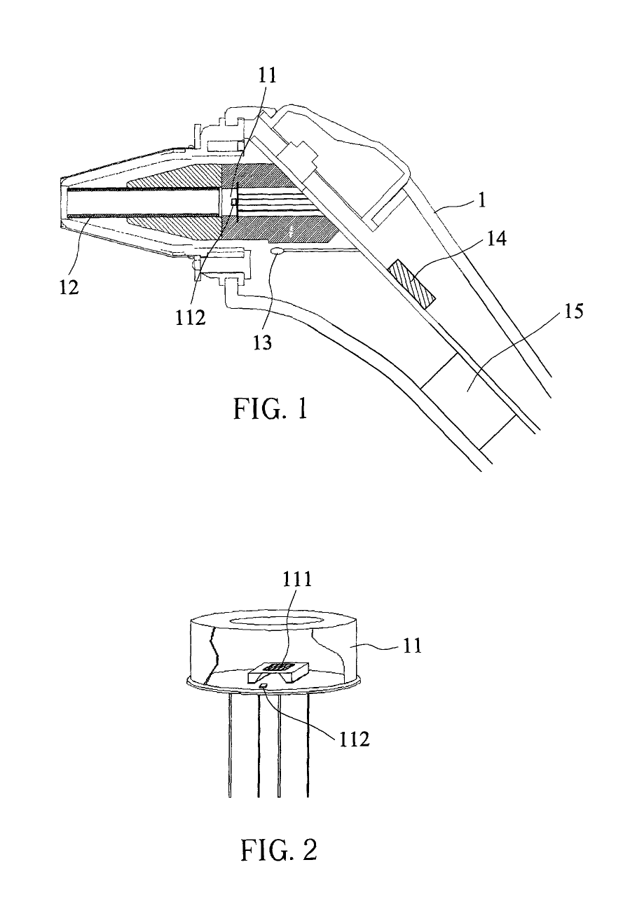

### DETAILED DESCRIPTION OF THE PREFERRED EMBODIMENTS

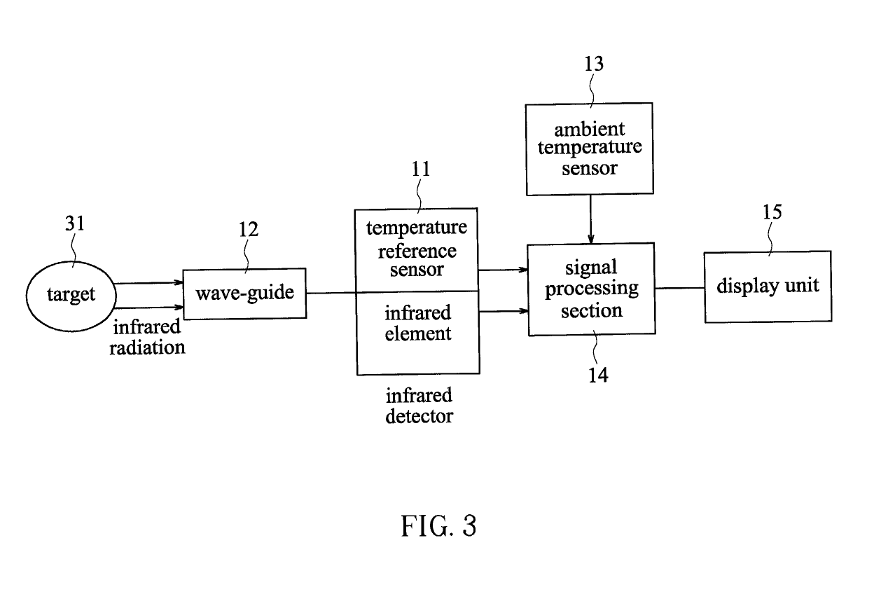

According to the one of embodiments' cross-sectional view of the present invention showing in FIG. 1, there are a housing 1, an infrared detector 11, an optical-guide 12, an ambient temperature sensor 13, a signal processing section 14, and a display unit 15. Wherein, optical-guide 12 is mounted with one end directed to collect infrared radiation from a measured target 31 as shown in FIG. 3. The other end of optical-guide 12 connects with infrared detector 11. Infrared detector 11 (as shown in FIG. 3) transmits the electric signal converted from infrared radiation to signal processing section 14. Ambient temperature sensor 13 is set in the space near the optical system (optical-guide 12 and infrared detector 11) to sense the ambient temperature change. Ambient temperature sensor 13 converts the ambient change into electric signal and transmits the signal to signal processing section 14 (as shown in FIG. 3). Display unit 15 is mounted on the housing to show the temperature reading after the signals from infrared detector 11 and ambient temperature sensor 13 are processed by signal processing section 14 (as shown in FIG. 3). In addition, infrared detector 11 comprises an infrared sensor element 111 and a temperature reference sensor 112, wherein temperature reference sensor 112 is used to detect the substrate 114 temperature of the infrared detector 11.

The following is the derivation of algorithm of the temperature offset method.

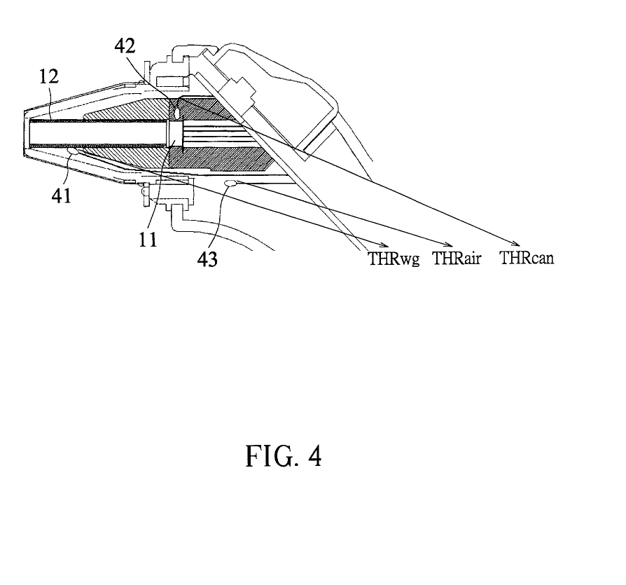

According FIG. 4, the test method with a test and experiment embodiment of the present invention that has three temperature reference sensors, THR_{wg}, THR_{can} and THRₐᵢᵣ. If the infrared radiation ear thermometer suffers from an ambient temperature change from Ta to Ta+T0, the temperature reference sensor THRₐᵢᵣ 43 in the space near the optical system (optical-guide 12 and infrared detector 11) is assumed to have a heat capacitance Cₐᵢᵣ and heat conductance Gₐᵢᵣ from the ambient. The heat capacity is defined by

Cₐᵢᵣ=dQ/dT (1)

Where dQ is the additional heat stored when its temperature is changed by an amount dT. The thermal conductance Gₐᵢᵣ is defined by the relation

P1=Gₐᵢᵣ*((T0+Ta)*u(t)−T(t)) (2)

Where P1 is the heat flow from the ambient to the thermometer and u(t) is the step function. The temperature of THRₐᵢᵣ 43 is governed by

Cₐᵢᵣ*dT(t)/dt=P1*u(t) (3)

Use (2) to replace P1

Cₐᵢᵣ*dT(t)/dt+Gₐᵢᵣ*T(t)=Gₐᵢᵣ*(T0+Ta)*u(t) (4)

Use Laplace transform to get the solution

Cₐᵢᵣ*(s*T(s)−Ta)+Gₐᵢᵣ*T(s)=Gₐᵢᵣ*(T0+Ta)/s (5)

From (4)

T(s)=Ta/(s+Gₐᵢᵣ/Cₐᵢᵣ)+((Ta+T0)*Gₐᵢᵣ/Cₐᵢᵣ)/(s*(s+Gₐᵢᵣ/Cₐᵢᵣ)) (6)

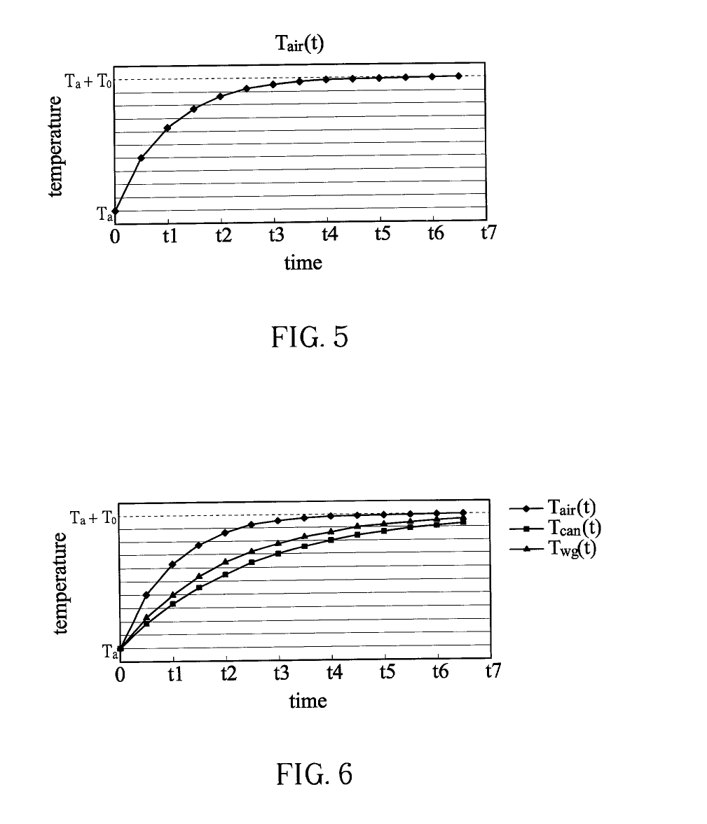

Let Gₐᵢᵣ/Cₐᵢᵣ=1/(τₐᵢᵣ), the solution of T(t) is derived from the inverse Laplace transform of (6) and drew in FIG. 5

Tₐᵢᵣ(t)=T0*(1−exp(−t/(τₐᵢᵣ)))+Ta (7)

Where τₐᵢᵣ is the thermal time constant of the space near the optical system (THRₐᵢᵣ 43) that can be derived from experiment and test.

The following is the description of the test method with a test and experiment embodiment (as shown in FIG. 4) that has three temperature reference sensors, THR_{wg}, THR_{can} and THRₐᵢᵣ, mounted separately in three different positions: (1) the front end of the optical-guide 12 (THR_{wg} designated as 41); (2) the metal can wall of the infrared detector 11 (THR_{can} designated as 42); (3) the space near the optical system (optical-guide 12 and infrared detector 11) of the thermometer (THRₐᵢᵣ designate as 43).

First, put the thermometer into an 18° C. (Tₐ) constant temperature chamber for at least 1hours to establish a thermal equilibrium condition. Second, take the thermometer out from the 18° C. chamber. Then, place it into another constant temperature chamber at 28° C. (Tₐ+T₀) immediately. The humidity of the chambers must be watched out to avoid the condensation. Record the changes of temperature from all temperature reference sensors, THR_{wg} 41, THR_{can} 42 and THRₐᵢᵣ 43, for 1 hour per sampling rate of 2-20 times per sec. The changes are drawn as temperature changing curves as shown in FIG. 6. From the changes, the thermal time constants of temperature reference sensors, THR_{wg} 41, THR_{can} 42 and THRₐᵢᵣ 43, are derived and represented as τ_{wg} and τ_{can} and τₐᵢᵣ respectively.

Because Tₐᵢᵣ(t), τₐᵢᵣ and Tₐ are known variables, the rapid ambient temperature change To can be calculated from formula (7). Apply the same theory to the positions near optical-guide 12 (Where the temperature sensor THR_{wg} 41 is positioned) and infrared detector metal can 113 (Where the temperature sensor THRcan 42 is positioned) where there are radiation exchanges with infrared detector element 111 when the thermometer is susceptible to the rapid ambient temperature change. We have the mathematical solution of the above temperatures.

T_{can}(t)=T0*(1−exp(−t/(τ_{can})))+Ta (8)

T_{wg}(t)=T0*(1−exp(−t/(τ_{wg})))+Ta (9)

Where T_{can}(t) and T_{wg}(t) are the temperatures of infrared detector metal can 113 and optical-guide 12, while τ_{can} and τ_{wg} are their thermal time constants respectively. The τ_{can} and τ_{wg} are also derived from the same experiment and test embodiment as the τₐᵢᵣ From formula (7), (8) and (9), the temperature of infrared detector 11 and optical-guide 12 are calculated even no real reference temperature sensor is placed on.

For real operation environment, the ambient changes are random both on direction and timing. Formula (4) must be rewritten as

Cₐᵢᵣ*dT(t)/dt+Gₐᵢᵣ*T(t)=Gₐᵢᵣ*((Ta+T0)*u(t)−(Ta+T0)*u(t−t1)+(Ta+T1)*u(t−t1)−(Ta+T1)*u(t−t2)+(Ta+T2)*u(t−t2)− (10)

Cₐᵢᵣ*dT(t)/dt+Gₐᵢᵣ*T(t)=Gₐᵢᵣ*((Ta+T0)*u(t)+Σ(n=1˜N)((Tn−Tn−1)*u(t−tn))) (11)

Where Tn may be positive or negative.

Use the Laplace transform and the principle of superposition, the temperature Tₐᵢᵣ(t) can be solved as

Tₐᵢᵣ(t)=Ta+T0*(1−exp(−t/(τₐᵢᵣ)))+Σ(n−1˜N)((Tn−Tn−1)*(1−exp(−(t−tn)/(τₐᵢᵣ)))*u(t−tn)) (12)

Where Tₐᵢᵣ(t), τₐᵢᵣ and Ta are known constant. T0˜Tn can be calculated if tn is also a known constant. The method to get the random timing tn will be described below.

First, consider a simple condition where the thermometer of the test and experiment embodiment (as shown in FIG. 4) suffers only one rapid temperature changes T0 at time t=0 as formula (7). Before time t=0, the thermometer is kept under an isothermal condition at temperature Ta. The signal processing section of the thermometer measures the temperature Tₐᵢᵣ(t) and its slope dTₐᵢᵣ(t)/dt constantly after the thermometer is powered on. The time derivative of Tₐᵢᵣ(t) is

dTₐᵢᵣ(t)/dt=(T0/τₐᵢᵣ)*exp(−t/τₐᵢᵣ) (13)

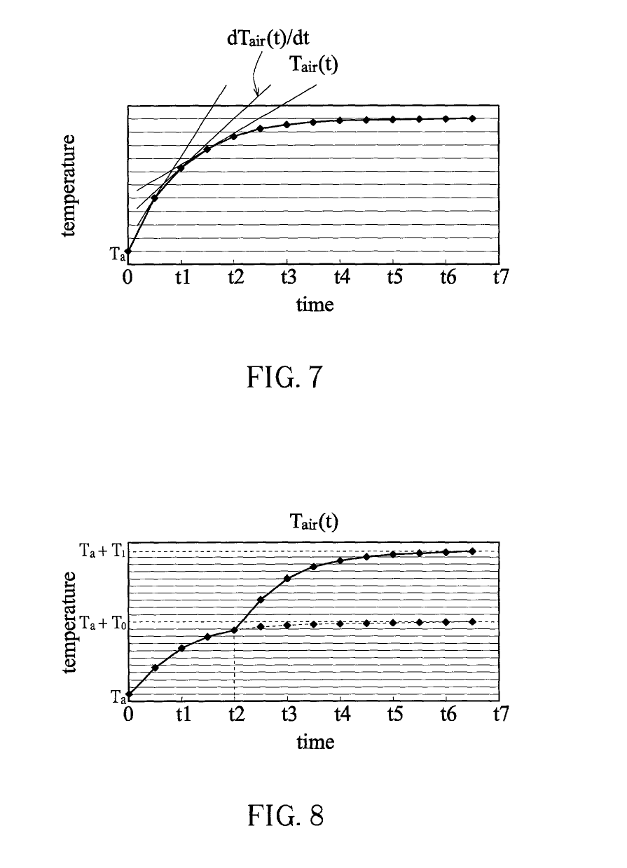

While the thermal time constant τₐᵢᵣ is smaller than τ_{can} and τ_{wg} and τₛₑₙ, (the time constant of the temperature reference sensor 112) the speed of the response and the detectability to the rapid temperature change of THRₐᵢᵣ 43 is faster and larger than THR_{can} 42, THR_{wg} 41 or THRₛₑₙ 112 at t=t0. That is why we use the time derivative of Tₐᵢᵣ(t) as the criterion of judging whether the ambient temperature of the thermometer is changing too fast or not. When the slope of Tₐᵢᵣ(t) is larger than a predetermined value (say 3/τₐᵢᵣ), the thermometer must be susceptible to a rapid ambient temperature change and the time is set to t=0 as shown in FIG. 5. As time going from t=0, the slope of Tₐᵢᵣ(t) as shown in FIG. 7 is getting smaller and finally reaching to zero from formula (13) . When the time is larger than a predetermined value, say 10 times the thermal time constant τₐᵢᵣ, the slope of Tₐᵢᵣ(t) is closing to zero and another isothermal condition is achieved at temperature, Ta+T0, because all the temperature Tₐᵢᵣ(t), Tₛₑₙ, T_{can}(t) and T_{wg}(t) are the same as shown in FIG. 6. If at time t=t1 the ambient changes from Ta+T0 to Ta+T1, Tₐᵢᵣ(t) is governed by setting N=1 in formula (12).

Tₐᵢᵣ(t)=Ta+T0*(1−exp(−t/(τₐᵢᵣ)))+(T1−T0)*(1−exp(−(t−t1)/(τₐᵢᵣ)))*u(t−t1)) (14)

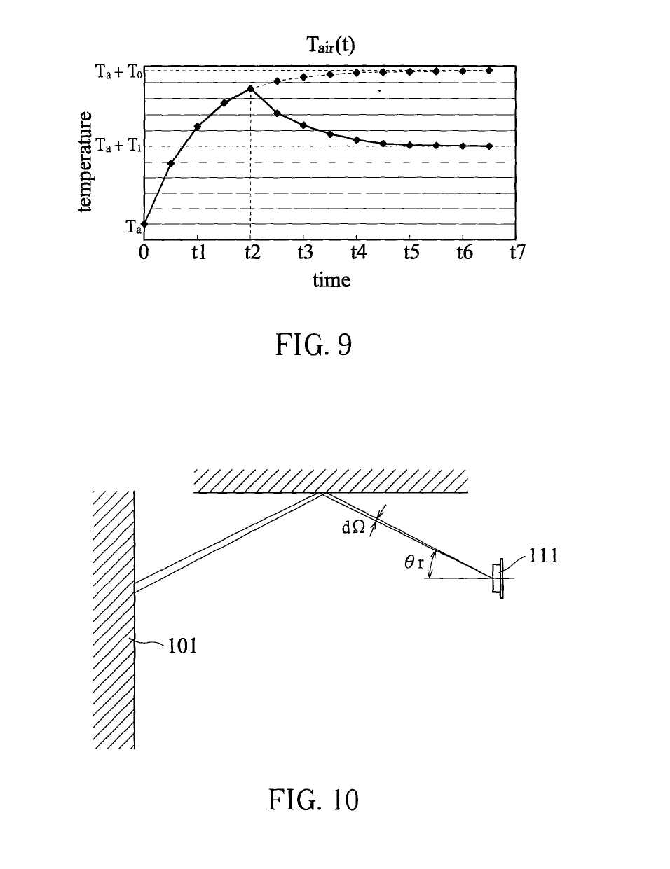

There are two cases should be taken into account. The first case is T1>T0 as shown in FIG. 8. The value of dTₐᵢᵣ(t)/dt will be increased at time t=t1 rather than decreasing as described above. The timer for 10 times τₐᵢᵣ will be reset and recount from zero. TI is calculated mathematically because Tₐᵢᵣ(t), Ta, T0 and τₐᵢᵣ are known. In the other case of T1<T0 (including T1<0), the sign of the slope of Tₐᵢᵣ(t) will change from positive to negative at t1 as shown in FIG. 9. For all the cases described above, whether and when (t0 . . . tn) the thermometer is susceptible to rapid temperature changes can be easily recognized from the time derivative of Tₐᵢᵣ(t) (the slope of Tₐᵢᵣ(t)). The amplitudes (T0 . . . Tn) of the ambient temperature interferences are also solved from the mathematical formula (12). Substitute τₐᵢᵣ by τ_{can} and τ_{wg}, the mathematical solutions of both T_{can}(t) and T_{wg}(t) are derived from formula (12). The effect on the accuracy of the thermometer of the temperature differences between Tₛₑₙ, T_{can} and T_{wg} under non-isothermal condition and the calculation of the offset will be discussed below.

As described in the above paragraph, the temperature of both metal can wall of the infrared detector 11 (T_{can}(t)) and optical-guide 12 (T_{wg}(t)) can be calculated mathematically from the temperature measured by a reference sensor (ambient temperature sensor 13 as shown in FIG. 1) near the optical system (optical-guide 12 and infrared detector 11) (Tₐᵢᵣ(t)), its time derivative (dTₐᵢᵣ(t)/dt) and the known time constant τ_{can} and τ_{wg} while both are derived from the above experiment.

First, consider the temperature difference between optical-guide 12 and infrared detector element 111.

As shown in FIG. 10, assume the irradiance dΦ from the target 101 with a radiance RA on the infrared detector element 111 with a small solid angle dΩ can be written as:

dΦ=RA*cos(θr)*dΩ (15)

From Stefan-Boltzmann law:

RA=(σTˆ 4/π)

We have

dΦ=(σTˆ 4/π)*cos(θr)*dΩ (16)

Where dΩ=2*π*sin(θr)dθr. Depend on the θr , the irradiance on the detector element 111 is reflected many times from the target 101 with the reflector (optical-guide 12, in FIG. 1). The times of reflection Nf is a function of θr:

Nf(θr)=floor((tan(θr)*Ssp+Rwg)/(2*Rwg)) (17)

Where Ssp is the distance from infrared detector element 111 to tip of the optical-guide 12, Rwg is the radius of the optical-guide 12.

Here we introduce the emissivity, reflection and the transmission into the equation to get the net irradiance on the infrared detector 11:

Φnet(θr, dT_{wg}, Tₜₐᵣ)=Φts(θr, Tₜₐᵣ)+Φws(θr, dT_{wg})−Φout(θr) (18)

Where Φts is the irradiance from target to detector, Φws is the irradiance from optical-guide 12, Φout is the radiation outgoing from infrared detector 11 and Tₜₐᵣ is the temperature of the target 101. These three items are derived from formulas (16) and (17) and written in detail:

$$Φ ts ( θ r , {T}_{tar} ) = 2 \cdot π \cdot {\int }_{0}^{θ rx} σ 1 \cdot cos ( θ r ) \cdot ϵ s \cdot {Ttar}^{4} \cdot {Rw}^{N ( θ r , d )} \cdot sin ( θ r ) ⅆ θ1 \qquad ( 19 )$$

Where θrx is FOV of the detector, σ1=σ*τd/π, τd is the transmission of the detector window, εs is the emissivity of the infrared detector element 111, Tₜₐᵣ is the target temperature and Rw=1−εw, the reflectivity of optical-guide 12 while εw is the emissivity of optical-guide 12.

$$Φ ws ( θ r , {dT}_{wg} ) = 2 π \cdot {\int }_{θ o}^{θ rx} σ1 \cdot cos ( θ r ) \cdot ϵ w \cdot ϵ s \cdot {( Tsen + dTwg )}^{4} \cdot {\sum }_{n = 1}^{Nf ( θ r , d )} {Rw}^{n - 1} \cdot sin ( θ r ) ⅆ θ t \qquad ( 20 )$$

Where θo=atan(Rwg/Ssp) is the maximum θr for zero reflection, dT_{wg} is the temperature difference between the sensor Tₛₑₙ and optical-guide 12 T_{wg}, that is T_{wg}=Tₛₑₙ+dT_{wg}.

$$Φ out ( θ r ) = 2 \cdot π \cdot {\int }_{0}^{θ rx} σ1 \cdot cos ( θ r ) \cdot ϵ s \cdot {Tsen}^{4} \cdot sin ( θ r ) ⅆ θ1 \qquad ( 21 )$$

The measurement error arose from the temperature difference between infrared detector 11 and optical-guide 12 can be written as:

dTₜₐᵣ/d(dT_{wg})=(dΦnet/d(dT_{wg}))/(dΦnet/dTₜₐᵣ) (22)

From formulas (18), (19), (20), (21), and (22) and the design values of the constants (the constants are different from one thermometer to another), we have:

dTₜₐᵣ/d(dT_{wg})=0.124 (23)

The measurement error, i.e. the value to be compensated from the measuring result arose from dT_{wg} is 0.124° K. per 1° K. difference between infrared detector 11 and the optical-guide 12.

Apply the same theorem to the metal can, if the temperature of metal can wall of the infrared detector 11 is T_{can}(t) which is different from the temperature of infrared detector element 111 Tₛₑₙ(t) under non-isothermal condition, the correcting value will be:

dTₜₐᵣ/d(dT_{can})=0.456 (24)

Where dT_{can}=T_{can}−Tₛₑₙ, is the temperature difference between the detector temperature and the can temperature.

Therefore, from the above description, the offset can be calculated with one temperature sensor, ambient temperature sensor 13 (THRₐᵢᵣ) instead of three temperature sensors, THR_{wg}, THR_{can} and THRₐᵢᵣ, mounted separately in three different positions: (1) the front end of the optical-guide 12 (THR_{wg} designated as 41); (2) the metal can wall of the infrared detector 11 (THR_{can} designated as 42). Consequently, the present invention can be realized by the preferred embodiment with only an ambient temperature sensor.

Then the derived offset is used to compensate the measured temperature reading under non-isothermal condition.

While the invention has been described by way of example and in terms of a preferred embodiment, it is to be understood that the invention is not limited to the disclosed embodiment. To the contrary, it is intended to cover various modifications. Therefore, the scope of the appended claims should be accorded the broadest interpretation so as to encompass all such modifications.

What we claim is:

1. An infrared ear radiation thermometer comprising:
an optical system for receiving and collecting infrared radiation from the ear canal of patient;
an infrared detector including an infrared sensor to convert infrared radiation energy collected through said optical system into an electrical signal, and a temperature reference sensor built in the infrared detector to sense the substrate temperature of the substrate of the infrared detector;
an ambient temperature sensor for detecting the ambient temperature change;
a signal processing section collecting electric signals from the infrared sensor, the reference temperature sensor and the ambient temperature sensor for producing a offset by an algorithm to eliminate the affecting of temperature change;
a display unit for showing a temperature reading;
wherein, the offset produced by signal processing section that computes the signals from said multiple temperature reference sensors is to maintain the infrared ear radiation thermometer in high precision and make the thermometer be used without waiting isothermal condition.

2. An infrared radiation thermometer offset method comprising:
a. Setting multiple sensors separately on the substrate of the infrared detector and in the space near the optical system to detect the difference between the ambient temperature and the infrared detector;
b. Transferring these detected signals from said multiple sensors to a signal processing section;
c. According these signals to produce an offset by an algorithm to compensate the thermometer reading error arose from the differences among the infrared detector, the optical system, and the ambient.

3. The infrared radiation thermometer offset method of claim 3, wherein setting using condition derives said algorithm from an experiment and test according to the real condition.
